(19) United States
(12) Patent Application Publication
(10) Pub. No.: US 2003/0124073 A1
(43) Pub. Date: Jul. 3, 2003

(54) Topical compositions containing solid particulates and a silicone resin copolymer fluid

(76) Inventors: Peter Jonathan Wyatt, Forest Hill, MD (US); Mannie Lee Clapp, Mason, OH (US); Colin Michael McHugh, Mason, OH (US); Rebecca Ann Taylor, Fairfield, OH (US); Claire Davison, Baltimore, MD (US); Karl Shiqing Wei, Mason, OH (US); Edward Dewey Smith III, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/255,282

(22) Filed: Sep. 26, 2002

Related U.S. Application Data
(60) Provisional application No. 60/325,022, filed on Sep. 26, 2001.

(51) Int. Cl. A61K 7/04; A61K 7/06; A61K 7/11

(52) U.S. Cl. 424/61; 424/70.12

(57) ABSTRACT

Disclosed are topical compositions for application to the hair, nails or skin, said compositions comprising (A) a solid particulate; and (B) an adhesive material in the form of a silicone resin copolymer as a condensation product of a polyalkylsiloxane and a silicone resin; and (C) an optional silicone-containing fluid, wherein the weight ratio of the adhesive silicone resin copolymer to the dispersed particulates is from about 5:1 to about 1:20, or wherein the average molecular weight of the adhesive silicone resin copolymer is at least about 15,000, or wherein the weight ratio of the optional silicone-containing fluid to the adhesive silicone resin copolymer is less than about 1.0. These defined copolymer characteristics provide the topical compositions with effective deposition and adherence of particulate solids onto the hair, skin or nails, while also providing extended wear cosmetics without reliance upon an excessively thick or heavy polymeric film to provide such benefits.

### CROSS REFERENCE TO RELATED APPLICATION

This application claims the benefit of U.S. Provisional Application No. 60/325,022, filed Sep. 26, 2001.

### FIELD OF THE INVENTION

The present invention relates to topical compositions containing solid particulates and an adhesive material in the form of a silicone resin copolymer fluid. The compositions provide extended wear characteristics as well as effective delivery and deposition of the solid particulates onto skin.

### BACKGROUND OF THE INVENTION

Topical compositions are applied to the skin for any of a variety of reasons, many of which include delivery of a pharmaceutical or skin active agent to the desired area of the skin. Although these topical compositions can be formulated in a variety of forms and include a nearly endless list of potential ingredients, these compositions often share a few common characteristics. For delivery of a pharmaceutical or skin active agent to the skin, the formulation must include a means for depositing the active on the skin or otherwise allowing the active sufficient exposure to the targeted area of the skin to provide the desired pharmaceutical or skin active effect.

Many pharmaceuticals or skin active agents do not readily or inherently adhere to and deposit onto the skin, so that such materials are often used in combination with a substantivity aid to trap or hold the active onto the skin for a time sufficient to provide the desired skin active effect. Examples of substantivity aids include silicone oils, film-forming or adhesive polymers, and many other functionally similar materials that provide substantivity benefits. Especially effective as substantivity aids in topical compositions are the various film forming polymers that help trap the active under or within a topical film or other continuous barrier from which the active is held on or near the skin and thus promotes active deposition. Examples of such film forming substantivity aids include certain silicone resin copolymers derived from the condensation of a polyalkylsiloxane with a silicone resin.

It has now been found that still other chemically distinct silicone resin copolymers when formulated as a fluid or liquid material within a topical composition are highly effective as substantivity aids when used in combination with a particulate solid, wherein; 1) the weight ratio of the silicone resin copolymer to the particulate solid is from about 5:1 to about 1:20, or 2) the average molecular weight of the copolymer is at least about 15,000, or 3) the composition contains a silicone-containing fluid such that the weight ratio of the silicone-containing fluid to the silicone resin copolymer is not more than about 1.0. These silicone resin copolymer compositions as defined herein provide excellent substantivity, extended wear characteristics, and highly desirable cosmetics and skin feel characteristics when applied topically to the skin from the compositions as defined herein.

It has been found that by using relatively high average molecular weight copolymers, or by formulating with a selected weight ratio of the copolymer to the dispersed particulates, that the above described benefits can be realized, provided that the silicone resin copolymer as formulated within the composition is solubilized or otherwise in liquid form. It has also been found that such benefits can also be achieved by formulating the composition with a silicone-containing fluid such that the weight ratio of the silicone-containing fluid to the silicone resin copolymer is not more than about 1.0.

It is therefore an object of the present invention to provide a topical formulation that provides effective delivery and deposition of a skin active agent onto skin, and further to provide such a composition that does not require the use of high silicone oil concentrations and does not leave an excessively heavy film on the applied areas of the skin. It is yet a further object of the present invention to provide such compositions using chemically distinct, adhesive, silicone resin copolymer fluids as defined herein.

### SUMMARY OF THE INVENTION

The present invention relates to topical compositions for application to the hair, nails or skin. These compositions comprise 1) solid particulates; 2) a silicone resin copolymer derived from the condensation of a polyalkylsiloxane and a silicone resin; and 3) an optional silicone-containing fluid, wherein the weight ratio of the silicone resin copolymer to the solid particulates is from about 5:1 to about 1:20, or wherein the average molecular weight of the silicone resin copolymer is at least about 15,000, or the weight ratio of the optional silicone-containing fluid to the adhesive silicone resin copolymer is less than about 1.0. The topical compositions provide effective deposition and adherence of solid particulates onto the skin while also providing extended wear properties, but without requiring the use excessively thick films on the applied surface to provide such substantivity and wear benefits. The topical compositions also provide desirable cosmetic or skin feel characteristics even though the compositions contains an adhesive material in the form of a silicone resin copolymer.

It has been found that by using relatively high average molecular weight silicone resin copolymers, or by formulating with a select weight ratio of the silicone resin copolymer to the solid particulates, that the above-described benefits can be realized. It has also been found that such benefits can also be achieved by formulating the composition with a silicone-containing fluid such that the weight ratio of the silicone-containing fluid to the silicone resin copolymer is not more than about 1.0.

### DETAILED DESCRIPTION

The topical compositions of the present invention comprise solid particulates and chemically distinct silicone resin copolymers. These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water. The topical compositions of the present invention can be aqueous or anhydrous.

The term “volatile” as used herein, unless otherwise specified, refers to those materials having an average boiling point at one (1) atmosphere of pressure (atm) of less than about 250° C., more typically less than about 235° C. at one (1) atm.

The term “ambient conditions” as used herein refers to surrounding conditions at one atmosphere of pressure, 50% relative humidity, and 25° C.

The term “copolymer fluid” as used herein, unless otherwise specified, refers to the silicone resin copolymer component of the compositions of the present invention, wherein the copolymer is solubilized in or otherwise in liquid form within the compositions. As such, the silicone resin copolymer material for use herein includes both solid and liquid materials, but when solid materials are used, the solid materials must be at least partially solubilized, preferably completely solubilized, within the composition and subsequently maintained within the composition in liquid form, all under ambient conditions.

All viscosity values as described herein, unless otherwise specified, are expressed in terms of centistokes (cs) and are determined or otherwise measure by a Brookfield DV-II+viscometer at 1 rpm at 25° C.

All percentages, parts and ratios as used herein are by weight of the total composition, unless otherwise specified. All such weights as they pertain to listed ingredients are based upon the active level and, therefore, do not include solvents or by-products that may be included in commercially available materials, unless otherwise specified.

The topical compositions of the present invention can comprise, consist of, or consist essentially of the essential elements and limitations of the invention described herein, as well as any additional or optional ingredients, components, or limitations described herein or otherwise useful in personal care compositions intended for topical application to the skin.

### Product Form

The topical compositions of the present invention can be formulated in any of a variety of product forms ranging from solids or semi-solids to liquids, and from rinse-off to leave-on applications. The key to all of the varied product forms contemplated within the scope of the compositions of the present invention is the defined combination of the particulate solids and the silicone resin copolymer as a condensation product of a polydialkylsiloxane and a silicone resin, all as defined herein.

The topical compositions of the present invention can be formulated in product forms suitable for us as color cosmetics, cosmetic foundations, rinse-off cleansers, or any other product form that provides for topical delivery of a skin active agent or benefit. Examples of such skin active agents or benefits, which will typically represent or be associated with part or all of the dispersed particulate solids as described hereinafter, are also described in greater detail hereinafter.

The topical compositions of the present invention can therefore be formulated and used as mascara, lip color cosmetics, cosmetic foundations, body cosmetics, sunscreens, moisturizing lotions, emollients, personal cleansing products, shampoos and other hair care products, pharmaceutical carriers, or any other product form in which the deposition and prolonged adherence of a solid particulate to the hair, nails or skin would be desirable.

### Silicone Resin Copolymer

The topical compositions of the present invention comprise a silicone resin copolymer derived from the condensation or other functionally similar reaction or combination of an organosiloxane resin with a diorganopolysiloxane fluid. These silicone resin copolymers are known for use as adhesives in various consumers' products and applications, and are now formulated into the compositions of the present invention for the purpose of improving the deposition or adherence of solid active particulates onto the hair, nails or skin.

The concentration of the silicone resin copolymer in the topical compositions of the present invention varies considerably depending upon other ingredients in the composition as well as the intended product form. Generally, silicone resin copolymer concentrations range from about 0.05% to about 40%, preferably from about 2% to about 35%, even more preferably from about 3% to about 35%, by weight of the topical composition. The concentration of the silicone resin copolymer is also selected so that 1) the ratio of the silicone resin copolymer to the solid particulates falls within the range as defined herein, and/or 2) the average molecular weight of the copolymer is as defined below, and/or 3) the weight ratio of any optional silicone-containing fluid to the silicone resin copolymer falls with the range as also defined herein.

The selected silicone resin copolymers for use in the topical compositions of the present invention preferably have an average molecular weight of at least about 15,000, more preferably from about 15,000 to about 4 million, even more preferably from about 100,000 to about 3 million.

It has been found that the preferred silicone resin copolymers as defined herein provide effective substantivity and extended cosmetic wear characteristics when formulated into the topical compositions of the present invention, regardless of whether they form a thick polymeric film over the applied surface area. It is believed that the selection of these particular silicone resin copolymers allows for improved active particle adherence and deposition to the hair, nail or skin, without the need for such heavy polymeric films over the applied areas to keep the solid active particulates deposited on and adhered to the intended site of application.

The silicone resin copolymers can be prepared by any known or otherwise effective method or chemistry for making such materials, non limiting examples of which include co-hydrolysis or by reacting triorganosilanes or other similar siloxanes with a silica hydrosol. The silicone resin copolymers are generally prepared by mixing and heating together an organosiloxane resin, diorganosiloxane fluid, and catalyst, at a temperature of above about 100° C., until the desired adhesive character of the resulting silicone resin copolymer is obtained. Mixing can be facilitated by the use of mutual solvents such as benzene, toluene, xylene, naptha, mineral spirits or other suitable solvent, which is subsequently removed from the mixture during the heating and mixing process.

The silicone resin copolymers for use in the topical compositions of the present invention are preferably prepared by heating a mixture of (1) from about 45% to about 75% by weight of the organosiloxane resin as a condensation product of SiO₂ and R₃(SiO)_{0.5} units, wherein each R group is independently selected from methyl, ethyl, propyl or vinyl radicals, and the ratio of SiO₂ units to R₃(SiO)_{0.5} units in the organosiloxane resin is from about 0.6 to about 1.0; (2) from about 25% to about 55% by weight of a hydroxyl end-blocked diorganopolysiloxane fluid having a viscosity in the range of from about 100 to about 100,000 cs at 25° C., wherein the organic substituents on the diorganosiloxane fluid are independently selected from methyl, ethyl, or vinyl radicals; and (3) from about 0.001% to about 5% by weight of a suitable catalyst, preferably an aliphatic organic amino compound selected from primary amines, secondary amines, tertiary amines, carboxylic acid salts of the above amines and quaternary ammonium salts. The mixture of materials is heated at a temperature above about 100° C. until the desired adhesive character of the resulting silicone resin copolymer is obtained.

Organosiloxane resins suitable for use in preparing the silicone resin copolymers for use herein are therefore those that preferably contain SiO₂ units and R₃(SiO)_{0.5} units (triorganosilyl) in a molecular ratio of from about 0.6 to about 1.0. Suitable triorganosilyl units for use in such organosiloxane resins include trimethylsilyl, triethylsilyl, methylmethylpropylsilyl, dimethylvinylsilyl, and combinations thereof. Preferred are trimethylsilyl units.

Diorganosiloxanes fluids suitable for use in preparing the silicone resin copolymers include hydroxyl end blocked diorganosiloxane polymers. The diorganosiloxanes are preferably linear polymers that contain only diorganosiloxane units, but can include small amounts of other materials such as triorganosiloxane units, monorganosiloxane units and SiO₂ units in minor amounts, typically less than about 1.0% by weight of the diorganosiloxane fluid, provided that the diorganosiloxane fluid remain hydroxyl end blocked to allow for the desired condensation reaction with the organosiloxane resin to form the silicone resin copolymer.

The organic substituents on the diorganosiloxane fluids for use in preparing the silicone resin copolymer can be any one or more of methyl, ethyl or vinyl radicals. Non-limiting examples of suitable diorganosiloxane fluids include ethylmethylpolysiloxane, copolymers of dimethylsiloxane and methylvinylsiloxane units, and mixtures of polymers or copolymers so long as such materials are hydroxyl end blocked. The viscosity of the diorganosiloxane polymer is preferably at least about 100 centistokes, and typically at least about 100,000 centistokes, as measured at 25° C., although it is understood that the diorganosiloxane fluid for use in this context can be substituted with a diorganosiloxane solid having a viscosity well beyond 10 million centistokes as measured at 25° C.

The organic amino compound for use as a catalyst in preparing the silicone resin copolymer includes any aliphatic hydrocarbon amine; alkanol amine; carboxylic acid salt thereof; and tertiary amine such as trimethylamine, tributylamine, methyldiproppylamine, and quaternary ammonium salts. This includes primary amines such as hexylamine, butanolamine, and butylamine; secondary amines such as diethylamine, diethanolamine, ethylamylamine and propylhexylamine; tertiary amines such as trimethylamine, tirbutylamine, methyldipropylamine, tirpropanolamine, and methylpropylhexylamine; and quaternary ammonium salts such as tetramethylammonium acetate and methylethyldibutylammoniumchloride, including quaternary ammonium emulsifying agents sold under various trade names, such as dioctadecyldimethylammonium chloride. In addition, any carboxylic acid salt of the amines, such as diethylamine acetate, butylamine octoate and trimethylamine laurate can be used. Tertiary amines are preferred, especially tertiary aliphatic amines.

### Solid Particulates

The compositions of the present invention comprise solid particulates dispersed throughout the composition, which are then deposited onto the hair, nails or skin after topical application of the composition. The solid particulates can be any skin active agent or other material known for or otherwise useful in application to and deposition on the skin. These solid particulates include materials such as emollients, moisture or oil absorbing solids, perfumes, vitamins, sunscreens, pigments or colorants, pharmaceuticals or other skin active agents, or any other solid material that provides a cosmetic, skin active, or other consumer desirable benefit when applied to and deposited on the skin.

It has been found that the solid particulates can be more effectively deposited onto the skin when used in combination with the silicone resin copolymer as described herein. It has been found that this combination results in a more effective deposition or adherence of the solid particulates to the applied area such that the solid particulates remain on the skin for prolonged periods of time. It has also been found that this combination can provide these benefits without also needing to form an excessively heavy polymeric film on the skin.

The weight ratio of the silicone resin copolymer to the solid particulates is preferably selected with a range of from about 5:1 to about 1:20, more preferably from about 4:1 to about 1:15. It has been found that this ratio provides effective deposition or adherence of the solid particulates while minimizing the development of a cosmetically unpleasant film over the applied surface area. The concentration of the solid particulates in the composition will vary considerably depending upon variables such as the desired product form, the silicone resin copolymer concentration, the type of solid particulate selected and its intended benefit, and other variables.

Other solid particulates suitable for use herein include any solid organic or inorganic color or pigment, or other appearance enhancer, suitable for topical application to the skin that is also insoluble in the selected product matrix or otherwise remains at least partially in the form of dispersed or suspended solids within the matrix. The solid pigment as a particulate solid is preferably selected from titanium oxide, iron oxide, chalk, talc, iron oxides, titanated micas, and combinations thereof. More preferred among colored or pigmented solids are titanium oxide, iron oxide, and combinations thereof.

Still other solid particulates suitable for use herein include solid or semi-solid emollients or skin feel agents, non-limiting examples of which include silicone elastomer solids or gels, silica-containing solids, plastic or other functionally similar polymeric solids, and similar other materials.

Other solid particulates suitable for use herein include perfume containing-particulates (e.g., encapsulated or other similar perfume systems) and solid particulates containing pharmaceutical or skin active agents such as antimicrobials (e.g., anti-fungal, anti-bacterial, anti-viral), steroidal anti-inflammatory agents, deodorant actives, preservatives (e.g., propylparaben), humectants, astringents, sensates (e.g., menthol, camphor), vitamins (e.g., Vitamin B complex, Vitamin C, Vitamin D, Vitamin E, Vitamin A), nutrients, analgesics, anesthetics, antihistamines, vasodilators, moisturizers, anti-aging and anti-wrinkle agents, and other pharmaceutical actives suitable for topical application with the intended topical or systemic effect.

### Absorbent Powders

The topical compositions of the present invention include those embodiments that contain absorbent powders as the solid particulate. The absorbent powder can be any solid material that provides moisture, sweat or sebum absorption when applied topically to the hair, nails or skin, and can be used in place of or in combination with other solid particulates as described hereinbefore. It is highly preferred that the absorbent powder have a Moisture Absorption Value of at least 1.0 gram/gram in accordance with the Moisture Absorbency Test as defined hereinafter.

The absorbent powders suitable for use as particulate solids herein are most typically in the form of porous, absorbent materials such as silicas (or silicon dioxides), silicates, carbonates, various organic copolymers, and combinations thereof. The silicates are most typically those formed by the reaction of a carbonate or silcate with an alkali metal, alkaline earth metal, or transition metal, specific non-limiting examples of which include calcium silicate, amorphous silicas, calcium carbonate, magnesium carbonate, zinc carbonate, and combinations thereof. Non-limiting examples of some suitable silicates and carbonates for use herein are described in Van Nostrand Reinhold's Encyclopedia of Chemistry, 4^{th} edition, pages 155, 169, 556, and 849 (1984), which descriptions are incorporated herein by reference. Absorbent powders are also described in U.S. Pat. No. 6,004,584 (Peterson et al.), which description is incorporated herein by reference.

Other absorbent powders suitable for use herein include kaolin, mica, talc, starch, modified starch, microcrystalline cellulose (e.g., Avicel from FMC Corporation), or other silica-containing or non-silica-containing powder suitable for absorbing fluids from the applied surface of the body.

Among the absorbent powders for use herein, highly preferred are those powders that have a Moisture Absorption Value of at least about 1.0, more preferably from about 2.0 to about 8.0, even more preferably from about 3.0 to about 7.0, grams of moisture absorption per gram of absorbent powder in accordance with the Moisture Absorption Method as described hereinafter. These Moisture Absorption Values have been found to correlate with the ability of the compositions of the present invention containing such materials to provide moisture, sweat and/or sebum absorption over extended periods of time after topical application.

It has been found, therefore, that the absorbent powders as described herein can be formulated into the compositions of the present invention to provide long lasting or enduring moisture, sebum and/or sweat absorption after application. It is believed that the silicone resin copolymer material as described herein provides improved deposition and adherence of the absorbent powders to the skin, even when such powders are formulated into liquid compositions, and even when formulated into rinse-off cleansing compositions, to thus provide long lasting or enduring absorption benefits on the applied area of the body.

### Moisture Absorption Test

The compositions of the present invention preferably contain a material, most typically an absorbent powder or solid, that provides a high rate of moisture absorption when applied topically to the skin. These materials can be characterized in terms their Moisture Absorption Value as measured in accordance with the following Moisture Absorption Test.

A powder chamber (Kruss Fiber Cell) and two filter papers (Kruss filter paper, part # FL12PLP) are preweighed on a balance and balance tared. One of the filter papers is then placed at the bottom of the powder chamber. The sample of absorbent powder to be tested is then packed into the chamber using a spatula. The mass of powder loaded into the chamber will vary depending upon the density of the powder, but will most typically range from about 0.50 grams to about 3.5 grams of powder packed into the powder chamber. A second filter paper is then placed on top of the packed powder and the screw cap for the chamber is placed on the chamber. The chamber knob is then rotated by hand until the powder is firmly packed and the knob can no longer be rotated manually. The powder chamber is then placed within a Kruss Tensiometer. The Tensiometer glass dish is then filled with distilled water and positioned on the stage device. The Tensiometer is then turned on and the stage is raised to just below the powder chamber so that the powder does not yet contact the distilled water in the dish. The Tensiometer is then balanced and allowed to tare. The Tensiometer is then turned off and the stage is raised until the powder chamber is immersed 9 mm in the distilled water. The mass of the powder sample is measured every fifteen seconds until the mass has reached equilibrium and no longer fluctuates drastically.

The Water Absorption Value is then determined for any given sample by calculating the difference between the mass of the powder chamber at 15 seconds and the mass of the powder chamber at the equilibrium point, divided by the mass of powder initially loaded into the chamber, all in accordance with the equation.

### Silicone Fluid

The cosmetic compositions of the present invention may further comprise a silicone-containing fluid, wherein the weight ratio of the silicone-containing fluid to the silicone resin copolymer is less than about 1.0, preferably less than about 0.5, more preferably from about 0.01 to about 0.05. It has been found that the silicone fluid can be formulated into the compositions of the present invention so as to provide these compositions with a smooth, non-sticky skin feel, provided also that the silicone fluid is formulated at the above-defined ratios relative to the silicone resin copolymer.

The silicone-containing fluids suitable for use in the compositions of the present invention can be any volatile or non-volatile silicone fluid that is suitable for topical application to the hair, nails or skin, and that is also compatible with the other selected ingredients of the composition. The concentration of the silicone fluid in the compositions preferably ranges from about 3% to about 15%, more preferably from about 5% to about 10%, even more typically from about 5% to about 8%, by weight of the composition.

The silicone-containing fluids for use herein include volatile and non-volatile diorganopolysiloxanes liquids that comprise repeating units corresponding to the formula (R₂SiO), where R is a monovalent hydrocarbon radical containing from 1 to 6 carbon atoms, preferably R is selected from methyl, ethyl, propyl, isopropyl, butyl, isobutyl, t-butyl, amyl, hexyl, vinyl, allyl, cyclohexyl, phenyl, fluoroalkyl and mixtures thereof. The fluid diorganosiloxane polymers may contain one or more of these hydrocarbon radicals as substituents on the siloxane polymer backbone. The diorganosiloxanes may be terminated by triorganosilyl groups of the formula (R′₃Si) where R′ is a radical selected from monovalent hydrocarbons containing from 1-6 carbon atoms, hydroxyl groups, alkoxyl groups and mixtures thereof. Polydimethylsiloxanes are preferred, especially linear dimethicones having a viscosity of from about 5 cs to about 500,000 cs, preferably from about 10 cs to about 200,000 cs, as measured at 25° C.

The silicone-containing fluids preferably include volatile silicone liquids, non-limiting examples of which are described in Todd et al., “Volatile Silicone Fluids for Cosmetics”, Cosmetics and Toiletries, 91:27-32 (1976). The volatile silicone fluid can be linear, cyclic or branched, but is preferably a cyclic silicone having from about 3 to about 7, more preferably from about 5 to about 6, silicon atoms, most preferably 5 silicon atoms. Among the volatile silicone fluids, cyclopentasiloxane is most preferred.

### Optional Ingredients

The topical compositions of the present invention may further comprise other optional ingredients that may modify the physical, chemical, cosmetic or aesthetic characteristics of the compositions or serve as additional “active” components when deposited on the skin. The compositions may also further comprise optional inert ingredients. Many such optional ingredients are known for use in personal care compositions, and may also be used in the topical compositions herein, provided that such optional materials are compatible with the essential materials described herein, or do not otherwise unduly impair product performance.

Such optional ingredients are most typically those materials approved for use in cosmetics and which are described in reference books such as the CTFA Cosmetic Ingredient Handbook, Second Edition, The Cosmetic, Toiletries, and Fragrance Association, Inc. 1988, 1992. Non limiting examples of such optional ingredients include preservatives, deodorants, antimicrobials, fragrances, deodorant perfumes, coloring agents or dyes, thickeners, antiperspirant active, sensates, sunscreens, gallants or other suspending agents, pH modifiers, co-solvents or other additional solvents, emollients, pharmaceutical actives, vitamins, and combinations thereof.

Other optional ingredients include surfactants, especially for those embodiments of the present invention intended for cleansing application to the hair, nails, or skin. Optional surfactant concentrations range from about 0.5% to about 20%, more typically from about 1% to about 10%, by weight of the topical composition. Suitable surfactants for optional use herein include cationic surfactants, anionic surfactants, non-ionionic surfactants, amphoteric surfactants, zwitterionic surfactants, and combinations thereof, that are suitable for topical application to the hair, nails or skin. Non-limiting examples of some suitable surfactants for use in the cleansing embodiments of the present invention are described in U.S. Pat. No. 6,113,890 (Young et. al.), which description is incorporated herein by reference. The cleansing embodiments of the present invention are therefore most typically used as rinse-off or wipe-off formulations.

Other optional ingredients include silicone elastomer powders and fluids to provide any of a variety of product benefits, including improved product stability, application cosmetics, emolliency, and so forth. The concentration of the silicone elastomers in the composition preferably ranges from about 0.1% to about 20%, more preferably from about 0.5% to about 10%, by weight of the composition. In this context, the weight percentages are based upon the weight of the silicone elastomers material itself, excluding any silicone-containing fluid that typically accompanies such silicone elastomers materials in the formulation process. The silicone elastomers suitable for optional use herein include emulsifying and non-emulsifying silicone elastomers, non-limiting examples of which are described in U.S. Ser. No. 09/613,266 (assigned to The Procter & Gamble Company), which description is incorporated herein by reference.

The optional ingredients as described herein shall specifically exclude, however, any essential ingredient or material as otherwise described or defined herein, so that the optional ingredients shall not include any material that is a solid particulate or silicone resin copolymer as defined herein.

### Method of Use

The topical compositions of the present invention are applied topically to the desired area of the hair, nails or skin in an amount sufficient to provide effective delivery of the desired solid particulate to the applied surface. The compositions can be applied to the desired area of the hair, nails or skin and allowed to remain, or the compositions can be rinsed or wiped off of the applied surface, especially for those surfactant-containing embodiments intended for application to and cleansing of the hair, nails or skin.

### Method of Manufacture

The topical compositions of the present invention may be prepared by any known or otherwise effective technique, suitable for making and formulating the desired product form. Specific non-limiting examples of such methods as they are applied to specific embodiments of the present invention are described in the following examples.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, i.e., weight/weight percentages, unless otherwise specified.

The topical compositions exemplified below provide deposition and adherence of various particulate solids to the hair, skin or nails. Each of the exemplified compositions provides extended, wear-resistant application of various particulate solids to the applied area of the hair, nails or skin.

Color Cosmetics

The following examples described in Table 1 are non-limiting examples of color cosmetic embodiments of the present invention.

TABLE 1
Color Cosmetics

| | Ex. | Ex. | Ex. | Ex |
|---|---|---|---|---|
| Ingredient | 1.1 | 1.2 | 1.3 | 1.4 |
| Adhesive solution | | | | |
| Cyclomethicone (DC245)¹ | 12.00 | 12.00 | 12.00 | 12.00 |
| Silicone Resin Copolymer² | 4.00 | 4.00 | 4.00 | 4.00 |
| Phase A | | | | |
| Cyclomethicone (DC245)¹ | 7.95 | 16.45 | 14.30 | 18.45 |
| Emulsifying elastomers gel (KSG21)³ | 5.00 | 5.00 | 5.00 | 2.50 |
| Propylparabens | 0.10 | 0.10 | 0.10 | 0.10 |
| Ethylparabens | 0.20 | 0.20 | 0.20 | 0.20 |
| Titanium dioxide | 8.25 | 8.25 | 9.90 | 8.25 |
| Iron oxide | 2.50 | 2.50 | 2.50 | 2.50 |
| Micronized Titanium dioxide (MT100TV)⁴ | — | — | 2.00 | — |
| Cetyl PEG/PPG-10/1 Dimethicone (Abil EM90)⁵ | — | — | — | 0.50 |
| Allyl methacrylates crosspolymer⁶ | — | — | — | 1.50 |
| Phase B | | | | |
| Water | 9.55 | 9.55 | 14.05 | 9.55 |
| Glycerin | 10.00 | 10.00 | 3.00 | 10.00 |
| Methylparabens | 0.10 | 0.10 | 0.10 | 0.10 |
| Disodium EDTA | 0.10 | 0.10 | 0.10 | 0.10 |
| Benzyl alcohol | 0.25 | 0.25 | 0.25 | 0.25 |
| Triethanolamine | — | — | 1.00 | — |
| Phenylbenzimidazole sulphonic acid⁷ | — | — | 1.50 | — |
| Phase C | | | | |
| Non-emulsifying elastomers gel (DC9040)⁸ | 40.00 | 30.00 | 30.00 | 30.00 |
| Total | 100 | 100 | 100 | 100 |

The compositions described above are prepared by conventional formulation and mixing techniques by first preparing the adhesive solution to form a 25% by weight solution of silicone resin copolymer in a cyclomethicone liquid. The Phase A ingredients are then combined under ambient conditions with the adhesive solution using a high sheer rotor/stator mixer to mix and deagglomerate the various ingredients until a homogeneous mixture is formed. The mixture is maintained in a warm water bath during the mixing process to the extent necessary to keep the mixture at room temperature.

In a separate vessel, the Phase B ingredients are mixed together and gently heated to the extent necessary to achieve complete dissolution resulting in a clear liquid. The clear liquid is cooled or allowed to cool to ambient temperature. The cooled liquid is then combined under ambient conditions with the Phase B mixture using a rotor/stator mixer at high speed until the mixture forms a homogenous emulsion and all of the water in the mixture is fully incorporated therein. This particular combination is maintained during mixing to the extent necessary to maintain the mixture and resulting emulsion at room temperature. The resulting emulsion is then mixed with the Phase C ingredients using a high sheer rotor/stator mixer until a homogenous solution forms.

The homogenous solution is then poured and packaged into a suitable package or applicator. Each of the homogenous solutions described above are color cosmetic embodiments of the present invention.

Liquid Cosmetics

The following examples described in Table 2 are non-limiting examples of liquid cosmetic embodiments of the present invention.

TABLE 2
Liquid Cosmetics

| | Ex. | Ex. | Ex. |
|---|---|---|---|
| Ingredient | 2.1 | 2.2 | 2.3 |
| Adhesive solution | | | |
| Cyclomethicone (DC245) | 12.00 | 12.00 | 12.00 |
| Silicone Resin Copolymer | 4.00 | 4.00 | 4.00 |
| Phase A | | | |
| Cyclomethicone (DC245) | 1.00 | 3.31 | 2.31 |
| Dimethicone copolyol (DC5225C) | 17.94 | 12.66 | 13.66 |
| Cetyl dimethicone (Abil Wax 9801) | — | — | 1.00 |
| Cetyl dimethicone copolyols (Abil EM90) | 0.20 | — | — |
| Dimethicone liquid | 4.00 | — | — |
| Micronized titanium dioxide (MT100TV) | 0.25 | 0.25 | 4.00 |
| Titanium dioxide | 8.25 | 8.25 | 8.00 |
| Iron oxide | 2.00 | 2.50 | 2.50 |
| Talc | 1.51 | 1.51 | — |
| Silica | 0.10 | 0.50 | 0.50 |
| Aluminum starch octenylsuccinate | 3.00 | — | — |
| Mica | — | 0.10 | 0.10 |
| Phase B | | | |
| Arachidyl behenate | 0.30 | 0.30 | 0.20 |
| Synthetic wax | 0.10 | 0.10 | — |
| Trihydroxystearin | 0.30 | 0.30 | 0.30 |
| Phase C | | | |
| Propylparaben | 0.25 | 0.25 | 0.25 |
| Laureth-7 | 0.50 | 0.50 | 0.50 |
| Ethylene brassylate | 0.05 | 0.05 | — |
| Phase D | | | |
| Water | 33.83 | 40.77 | 38.03 |
| Propylene glycol | 7.00 | — | — |
| Sodium chloride | 2.00 | 2.00 | 2.00 |
| Methylparaben | 0.12 | — | |
| Sodium dehydroacetate | 0.30 | 0.30 | 0.30 |
| Polyvinylpyrollidone (PVP) | 1.00 | — | |
| Glycerin | — | 10.00 | 10.00 |
| Trisodium EDTA | — | 0.10 | 0.10 |
| Phenoxymethanol | — | 0.25 | 0.25 |
| Total | 100 | 100 | 100 |

The liquid cosmetic embodiments described in Table 2 are prepared by conventional formulation and mixing techniques by first preparing the adhesive solution to form a 25% solution of silicone resin copolymer in a cyclomethicone liquid. The Phase A ingredients are then combined under ambient conditions with the adhesive solution and subjected to high sheer rotor/stator mixing to deagglomerate the various ingredients until a homogeneous mixture is formed. The resulting homogenous mixture is then heated to 85° C. and combined with all of the Phase B ingredients other than trihydroxystearin until the Phase B ingredients have melted and dispersed, and then the heated mixture is cooled to 65° C. before adding trihydroxystearin (if present). The cooled mixture is mixed until all ingredients are fully dispersed, and then allowed to cool further to room temperature. The room temperature mixture is then combined with the Phase C ingredients until a homogenous intermediate mixture forms.

In a separate vessel, the Phase D ingredients are combined and mixed together until a clear phase forms, at which point the clear phase is added to the homogenous intermediate mixture above and emulsified using a rotor/stator mixer at high speed until the clear (aqueous) phase is fully incorporated into the resulting emulsion. The resulting emulsion is a liquid cosmetic embodiment of the present invention that is then packaged into appropriate containers and applicators.

Liquid Body Cosmetics

The following examples described in Table 3 are non-limiting examples of liquid body cosmetic embodiments of the present invention.

TABLE 3
Liquid Body Cosmetics

| | Ex. | Ex. | Ex. | Ex. | Ex. | Ex. | Ex. |
|---|---|---|---|---|---|---|---|
| Ingredients | 3.1 | 3.2 | 3.3 | 3.4 | 3.5 | 3.6 | 3.7 |
| Bio-PSA | 15.00 | 10.00 | 10.00 | 15.00 | 10.00 | 15.00 | 5.00 |
| Dimethicome Gum² | 0.00 | 7.50 | 0.00 | 0.00 | 5.00 | 5.00 | 5.00 |
| Bentone Gel³ | 27.39 | 27.39 | 34.20 | 31.50 | 31.50 | 20.00 | 30.00 |
| Total Solid Pigments | 25.00 | 15.00 | 15.00 | 20.00 | 20.00 | 20.00 | 20.00 |
| Anatasa TiO2 | 15.99 | 9.59 | 9.59 | — | — | — | — |
| Rutile TiO2 | — | — | — | 16.95 | 10.69 | 16.95 | 16.95 |
| Yellow Iron Oxide | 5.93 | 3.56 | 3.56 | 2.13 | 6.32 | 2.13 | 2.13 |
| Red Iron Oxide | 2.61 | 1.57 | 1.57 | 0.77 | 2.21 | 0.77 | 0.77 |
| Black Iron Oxide | 0.48 | 0.29 | 0.29 | 0.15 | 0.78 | 0.15 | 0.15 |
| Propylparaben | 0.20 | 0.20 | 0.20 | 0.20 | 0.20 | 0.20 | 0.20 |
| Isododecane | 32.41 | 39.91 | 39.60 | 33.30 | 33.30 | 38.80 | 36.80 |
| Elastomer | 0.00 | 0.00 | 1.00 | 0.00 | 0.00 | 1.00 | 3.00 |

Lipcolor Cosmetics

The following examples described in Table 4 are non-limiting examples of lipcolor cosmetic embodiments of the present invention.

TABLE 4
Lipcolor Products

| | Ex. | Ex. | Ex. | Ex. |
|---|---|---|---|---|
| Ingredient | 4.1 | 4.2 | 4.3 | 4.4 |
| Phase A | | | | |
| Titanium dioxide | 3.00 | 2.00 | 3.00 | 3.00 |
| Iron oxide | 2.00 | 1.33 | 2.00 | 2.00 |
| Titanated mica | 10.00 | 6.67 | 10.00 | 10.00 |
| Isododecane | 15.00 | 15.00 | 15.00 | 15.00 |
| Propylparaben | 0.30 | 0.30 | 0.30 | 0.30 |
| Phase B | | | | |
| Elastomer gel (ShinEtsu KSG 32) | 33.99 | 38.99 | 23.99 | — |
| Bentone Clay Gel (Rheox Bentone | — | — | — | 33.99 |
| Gel ISD) | | | | |
| Phase C | | | | |
| Isododecane | 10.71 | 10.71 | 13.71 | 10.71 |
| Silicone Resin Copolymer | 25.00 | 25.00 | 32.00 | 25.00 |
| Total | 100.00 | 100.00 | 100.00 | 100.0 |

The liquid cosmetic embodiments described in Table 4 are prepared by conventional formulation and mixing techniques by first preparing Phase C to form a 70% solution of silicone resin copolymer in isododecane. The Phase A ingredients are then combined under ambient conditions by first mixing and dissolving the trimethylsiloxysilicate (an example of a material which can act as a dispersing aid for some colorants) with the isododecane. Then the colorants and preservative (Propylparaben) are added. The mixture is then subjected to mixing using a lab bench propeller mixer to wet the solids with the liquid. Then the mixture is subjected to high sheer rotor/stator mixing to deagglomerate the various ingredients until a homogeneous mixture is formed. The resulting homogenous mixture is combined at ambient conditions with phase B and mixed until the resulting mixture is homogeneous. The room temperature mixture is then combined with Phase C and then mixed until homogeneity, again at ambient conditions. The resulting lipcolor is a liquid cosmetic embodiment of the present invention that is then packaged into appropriate containers and applicators.

What is claimed is:

1. Topical compositions for application to the hair, nails or skin, said compositions comprising:
(A) solid particulates; and
(B) a silicone resin copolymer as a condensation product of a diorganosiloxane fluid and a silicone resin;
wherein the weight ratio of the silicone resin copolymer to the solid particulates is from about 5:1 to about 1:20.

2. The compositions of claim 1 wherein the weight ratio of the silicone resin copolymer to the solid particulates is from about 4:1 to about 15:1.

3. The composition of claim 1 wherein the silicone resin copolymer has an average molecular weight of at least about 15,000.

4. The composition of claim 1 wherein the silicone resin copolymer has an average molecular weight of from about 100,000 to about 3 million.

5. The composition of claim 1 wherein the diorganosiloxane fluid is a hydroxyl end blocked diorganopolysiloxane having a viscosity of from about 100 to about 100,000 cs at 25° C., wherein the organic substituents on the diorganopolysiloxane fluid are selected from the group consisting of methyl, ethyl, and vinyl radicals.

6. The composition of claim 1 wherein the silicone resin is as a condensation product of SiO₂ and R₃(SiO)_{0.5} units; wherein each R group is independently selected from the group consisting of methyl, ethyl, propyl and vinyl radicals; and the molar ratio of SiO₂ units to R₃(SiO)_{0.5} units in the silicone resin is from about 0.6 to about 1.0.

7. The composition of claim 1 wherein the composition further comprises a silicone-containing fluid.

8. The composition of claim 7 wherein the silicone-containing fluid comprises a dimethicone fluid, and the weight ratio of the silicone-containing fluid to the silicone resin copolymer is less than about 1.0.

9. The composition of claim 8 wherein the weight ratio of the silicone-containing fluid to the silicone resin copolymer is from about 0.01 to about 0.5.

10. The composition of claim 7 wherein the silicone-containing fluid comprises cyclopentasiloxane.

11. The composition of claim 1 wherein the solid particulates comprise moisture-absorbing solids.

12. The composition of claim 10 wherein the moisture-absorbing solids are selected from the group consisting of silicates, carbonates, organic copolymers, and combinations thereof.

13. The composition of claim 11 wherein the moisture-absorbing solid has a Moisture Absorption Value of at least about 1.0 grams/gram.

14. The composition of claim 11 wherein the Moisture Absorption Value ranges from about 3.0 grams/gram to about 8.0 grams/gram.

15. The composition of claim 1 wherein the silicone resin copolymer represents from about 2% to about 40% by weight of the composition.

16. The composition of claim 1 wherein the composition further comprises from about 0.1% to about 20% by weight of a silicone elastomer.

17. The composition of claim 1 wherein the solid particulates comprise solid pigments

18. The composition of claim 1 wherein the composition is a leave-on composition.

19. Topical compositions for application to the hair, nails or skin, said compositions comprising:
(A) solid particulates;
(B) a silicone resin copolymer as a condensation product of a polyalkylsiloxane and a silicone resin; and
(C) a silicone-containing fluid;
wherein the weight ratio of the silicone-containing fluid to the silicone resin copolymer is less than about 1.0.

20. The compositions of claim 19 wherein the weight ratio of the silicone resin copolymer to the solid particulates is from about 5:1 to about 1:20.

21. The composition of claim 19 wherein the silicone resin copolymer has an average molecular weight of at least about 15,000.

22. The composition of claim 19 wherein the silicone resin copolymer has an average molecular weight of from about 100,000 to about 3 million.

23. The composition of claim 19 wherein the wherein the weight ratio of the silicone-containing fluid to the silicone resin copolymer is from about 0.01 to about 1.0.

24. The composition of claim 19 wherein the diorganosiloxane fluid is a hydroxyl end blocked diorganopolysiloxane having a viscosity of from about 100 to about 100,000 cs at 25° C., wherein the organic substituents on the diorganopolysiloxane fluid are selected from the group consisting of methyl, ethyl, and vinyl radicals.

25. The composition of claim 19 wherein the silicone resin is as a condensation product of SiO₂ and R₃(SiO)_{0.5} units; wherein each R group is independently selected from the group consisting of methyl, ethyl, propyl and vinyl radicals; and the molar ratio of SiO₂ units to R₃(SiO)_{0.5} units in the silicone resin is from about 0.6 to about 1.0.

26. The composition of claim 19 wherein the silicone-containing fluid represents from about 3% to about 15% by weight of the composition and comprises a dimethicone fluid.

27. The composition of claim 19 wherein the silicone-containing fluid comprises cyclopentasiloxane.

28. The composition of claim 19 wherein the solid particulates comprise moisture-absorbing solids.

29. The composition of claim 28 wherein the moisture-absorbing solids are selected from the group consisting of silicates, carbonates, organic copolymers, and combinations thereof.

30. The composition of claim 29 wherein the absorbing-absorbing solid has a Moisture Absorption Value of at least about 1.0 grams/gram.

31. The composition of claim 30 wherein the Moisture Absorption Value ranges from about 3.0 grams/gram to about 8.0 grams/gram.

32. The composition of claim 19 wherein the silicone resin copolymer represents from about 2% to about 40% by weight of the composition.

33. The composition of claim 19 wherein the composition further comprises from about 0.1% to about 20% by weight of a silicone elastomer.

34. The composition of claim 19 wherein the solid particulates comprise solid pigments.

35. The composition of claim 19 wherein the composition is a leave-on composition.

36. Topical compositions for application to the hair, nails or skin, said compositions comprising:
(A) solid particulates; and
(B) a silicone resin copolymer as a condensation product of a polyalkylsiloxane and a silicone resin;
wherein the average molecular weight of the silicon resin copolymer is at least about 15,000.

37. The compositions of claim 36 wherein the weight ratio of the silicone resin copolymer to the solid particulates is from about 5:1 to about 1:20.

38. The composition of claim 37 wherein the silicone resin copolymer has an average molecular weight of at least about 15,000.

39. The composition of claim 36 wherein the silicone resin copolymer has an average molecular weight of from about 100,000 to about 3 million.

40. The composition of claim 36 further comprising a silicone-containing fluid at a weight ratio of the silicone-containing fluid to the silicone resin copolymer of less than about 1.0.

41. The composition of claim 36 wherein the diorganosiloxane fluid is a hydroxyl end blocked diorganopolysiloxane having a viscosity of from about 100 to about 100,000 cs at 25° C., wherein the organic substituents on the diorganopolysiloxane fluid are selected from the group consisting of methyl, ethyl, and vinyl radicals.

42. The composition of claim 36 wherein the silicone resin is as a condensation product of SiO₂ and R₃(SiO)_{0.5} units; wherein each R group is independently selected from the group consisting of methyl, ethyl, propyl and vinyl radicals; and the molar ratio of SiO₂ units to R₃(SiO)_{0.5} units in the silicone resin is from about 0.6 to about 1.0.

43. The composition of claim 38 wherein the silicone-containing fluid represents from about 3% to about 15% by weight of the composition and comprises a dimethicone fluid.

44. The composition of claim 36 wherein the silicone-containing fluid comprises cyclopentasiloxane.

45. The composition of claim 37 wherein the solid particulates comprise moisture-absorbing solids.

46. The composition of claim 41 wherein the moisture-absorbing solids are selected from the group consisting of silicates, carbonates, and combinations thereof.

47. The composition of claim 41 wherein the absorbing-absorbing solid has a Moisture Absorption Value of at least about 1.0 grams/gram.

48. The composition of claim 43 wherein the Moisture Absorption Value ranges from about 3.0 grams/gram to about 8.0 grams/gram.

49. The composition of claim 36 wherein the silicone resin copolymer represents from about 2% to about 40% by weight of the composition.

50. The composition of claim 36 wherein the composition further comprises from about 0.1% to about 20% by weight of a silicone elastomer.

51. The composition of claim 36 wherein the solid particulates comprise color pigments.

52. The composition of claim 36 wherein the composition is a leave-on composition.
